SEQUENCE LISTING (2 sequences)

SEQ ID NO: 1 - DNA, 17 bp, Artificial
  PIVB-4 primer
tttggccgccgcccagt

SEQ ID NO: 2 - DNA, 18 bp, Artificial
  LMB10-E primer
gatggcgcccaacagtcc

(19) United States
(12) Patent Application Publication
(10) Pub. No.: US 2010/0304378 A1
(43) Pub. Date: Dec. 2, 2010

(54) Method for Detecting or Quantifying a Truncating Mutation

(75) Inventors: Andrew David Griffiths, Strasbourg (FR); Alex Garvin, Durmenach (FR); Kerstin Godelinde Blank, Leuven (BE)

Correspondence Address: SALIWANCHIK LLOYD & SALIWANCHIK;A PROFESSIONAL ASSOCIATION, PO Box 142950, GAINESVILLE, FL, 32614, US

(73) Assignees: Universite De Strasbourg, Strasbourg (FR); Centre National De La Recherche Scientifique, Paris, Cedex 16 (FR)

(21) Appl. No.: 12/675,542

(22) PCT Filed: Sep. 16, 2008
(86) PCT No.: PCT/EP2008/062325

§ 371 Date: Feb. 26, 2010

Related U.S. Application Data
(60) Provisional application No. 60/994,022, filed on Sep. 17, 2007.

(51) Int. Cl. C12Q 1/68; C12N 15/63

(52) U.S. Cl. 435/6; 435/320.1

(57) ABSTRACT

The present invention discloses a new method for detecting or quantifying a truncating mutation of a target gene in a subject, said method relying on the in vitro compartmentalization of single genetic constructs in aqueous droplets of a water-in-oil emulsion.

### FIELD OF THE INVENTION

The present invention relates to the field of medicine, in particular to the field of diagnosis. In particular, the present invention relates to clinical diagnosis based on gene mutation analysis.

### BACKGROUND OF THE INVENTION

All cancers are caused by genetic aberrations, i.e., gene mutations such as point mutations, insertions, frame shifts, deletions or translocations, which result in a modified expression level or an altered function of the mutated gene. These mutations can arise spontaneously or by external factors such as chemical mutagens, radiation, or viral integration.

Genetic abnormalities found in cancer typically affect two general classes of genes, oncogenes and tumor suppressor genes. Cancer-promoting oncogenes are typically activated in cancer cells, giving those cells new properties, such as hyperactive growth and division. Tumor suppressor genes are inactivated in cancer cells, resulting in the loss of normal functions in those cells, such as accurate DNA replication or control over the cell cycle.

In their earliest stages most cancers are clinically silent and patient diagnosis typically involves invasive procedures that are unpleasant for the patient or non-invasive procedures that frequently lack sensitivity and accuracy. The prognosis of cancer patients is most influenced by the type of cancer, as well as the stage or extent of the disease. Thus, the detection of early stage pre-cancerous growths will lead to improved treatment and patient outcome.

Colorectal cancer (CRC) is the third most common malignancy worldwide; in the year 2000 there were 945,000 new cases (9.4% of the world cancer total) and 492,000 deaths caused by CRC (7.9% of the world cancer total). This cancer can be prevented by detecting and removing polyps (or adenomas) from patients. Polyps are currently detected by colonoscopy, which is expensive and unpleasant, needing general anesthesia in some cases, and not without risk to the patient.

Regarding sensitivity and patient acceptance, detecting mutations in specific biomarkers in stool DNA seems to be a promising approach for early diagnostic of CRC.

Virtually all colorectal cancer tumours are derived from polyps and these tumours have the same APC mutations found in the polyps plus additional mutations in other genes (i.e., K-ras, P53, BAT-26). Moreover, DNA obtained from a stool sample of a patient with a polyp has a mutation in the Multiple Cluster Region (MCR) of the APC gene, a 1.2 Kb region in exon 15, in an estimated 83% of all cases (the other 17% of polyps presumably have mutations elsewhere in the APC gene).

Important efforts have been made to develop APC mutation detecting methods based on stool DNA but the best methods currently available offer a limited sensitivity. The first issue with such an approach is a technical one, because more than 99% of stool DNA will not have a mutation in the APC gene. The second issue is that mutations can be found anywhere in a 1,200 base pair region of the MCR for most polyps.

Well established methods for detecting low levels of mutant DNA, such as TaqMan Real-time PCR, allele specific PCR, the ligation chain reaction, and BEAMing (Li et al., 2006) are designed to detect a particular mutation at a particular base in a test gene and 1,200 independent assays would be needed to detect most of the clinically relevant APC mutations in a stool sample.

A number of groups have tried to detect polyps in patients by analyzing stool DNA but the currently available techniques allow detection of tumors and not polyps or only look for a small number of known APC mutations and do not attempt to scan the multiple cluster region of the APC gene.

Some other available techniques such as high-throughput sequencing (Thomas et al., 2006) or the method described by Traverso et al. for a high sensitivity electrophoresis based protein truncation test (Traverso, et al., 2002), could in principle be used to detect polyps from stool DNA, but the cost of these methods would be prohibitive for a clinical application.

Accordingly, there is a significant need for a high-sensitive, non-invasive and cost-effective method to detect disease causing mutations and thus to diagnose or prognosticate a disease, particularly a cancer.

### SUMMARY OF THE INVENTION

In a first aspect, the present invention provides a method for detecting or quantifying a truncating mutation of a target gene in a subject, said method relying on the in vitro compartmentalization of single genetic constructs in aqueous droplets of a water-in-oil emulsion and comprising:

a) providing a DNA sample from the subject;

b) assembling genetic constructs, each construct comprising a test sequence of said target gene, obtained from said DNA sample, operably linked with a promoter and a ribosome binding site, a first reporter system used to control the presence of said test sequence in the construct and/or the presence of a construct comprising said test sequence in a droplet, and a second reporter system used to detect the presence of a truncating mutation of said target gene, said first and second reporter systems generating distinct signals;

c) compartmentalizing each genetic construct in a droplet by forming a water-in-oil emulsion;

d) transcription and translation of each genetic construct in each droplet;

e) monitoring emitted signals from said first and second reporter systems in each droplet to detect or quantify truncating mutations of said target gene.

In a second aspect, the present invention provides a method for detecting or quantifying a truncating mutation of a target gene in a subject, said method relying on the in vitro compartmentalization of single genetic constructs in aqueous droplets of a water-in-oil emulsion and comprising:

a) providing a DNA sample from the subject;

b) compartmentalizing each DNA molecule, from said DNA sample, comprising a test sequence of said target gene in first droplets by forming a water-in-oil emulsion;

c) assembling genetic constructs, each construct comprising said test sequence operably linked with a promoter and a ribosome binding site, a marker gene of a first reporter system used to control the presence of said test sequence in the construct and/or the presence of a construct comprising said test sequence in a droplet, and a marker gene of a second reporter system used to detect the presence of a truncating mutation of said target gene, said first and second reporter systems generating distinct signals;

d) fusing first droplets containing genetic constructs of step c) with second droplets containing an in vitro transcription and translation system;

e) transcription and translation of each genetic construct in each fusion droplet

f) monitoring emitted signals from said first and second reporter systems in each fusion droplet to detect or quantify truncating mutations of said target gene.

In third aspect, the present invention provides a genetic construct comprising a test sequence operably linked with a promoter and a ribosome binding site, a first marker gene operably linked with another promoter, and a second marker gene which is operably linked to the test sequence in order to be expressed in a single mRNA, said marker gene being downstream to said test sequence.

In a fourth aspect, the present invention provides a genetic construct comprising a test sequence operably linked with a promoter and a ribosome binding site, a first reporter system which is an affinity system comprising two members, a first member appended on the 5′ end of the coding strand of the test sequence and a second member which is bound to the first member and is able to generate a signal, and a marker gene which is operably linked to the test sequence in order to be expressed in a single mRNA, said marker gene being downstream to said test sequence.

In another aspect, the present invention provides a kit for the detection or quantification of a truncating mutation in a target gene in a subject by using the method according to the invention, comprising at least reagents needed for the genetic construct assembling, an in vitro transcription/translation system, reagents needed for water-in-oil emulsion and, optionally means needed to compartmentalize each genetic construct or DNA molecule into droplets.

The present invention also provides a droplet from a water-in-oil emulsion containing a genetic construct comprising at least two reporter systems generating distinct signals.

### BRIEF DESCRIPTION OF THE DRAWINGS

FIG. 1 is a schematic representation of one embodiment of the method according to the invention as detailed in the experimental section (example 2).

FIG. 2 is a schematic representation of one embodiment of the method according to the invention as detailed in the experimental section (example 3)

FIG. 3 is a graph showing fluorescence analysis of 100,000 emulsion PCR droplets. Droplets fluorescence was monitored using a 488 nm laser and a photomultiplier tube (PMT) to collect the light emitted at 525 nm.

FIG. 4 shows fluorescence analysis of emulsion HRCA droplets obtained with various DNA concentrations: FIG. 4A, λ=0; FIG. 4B, λ=0.06; FIG. 4C, λ=1.28; FIG. 4D, λ=10

FIG. 5 is a schematic representation of the droplet fusion device. The droplet fusion device consists of four separate modules integrated into single microfluidic chip, that is, (i) emulsion reinjection, (ii) on-chip droplets generation, (iii) droplets pairing and (iv) electro-coalescence modules. Numbers inside the graph represent: (1) droplet reinjection inlet, (2) carrier oil inlet used to space reinjected droplet, (3) IVT aliquot inlet, (4) carrier oil inlet used to produce IVT droplets, and (5) collection outlet.

FIG. 6 shows fluorescence analysis of emulsion IVT-HRCA fused droplets. HRCA reaction was carried out using a DNA concentration corresponding to λ=0.25, in absence (left) or presence (right) of Phi29 DNA polymerase. Both emulsions contained dextran-texas red conjugate. After amplification, HRCA droplets were fused with IVT/FDG containing droplets, incubated and reinjected on analysis device. Droplets fluorescence was monitored using a 488 nm and 532 nm lasers (exciting fluorescein and texas red respectively) and photomultiplier tubes (PMT) to collect the light emitted at 525 nm and 610 nm (for fluorescein and texas red respectively). The identity of the different populations is given and the percentage of the total population is indicated. A single fused IVT droplet corresponds to 1 HRCA droplet fused with 1 IVT droplet and a double-fused droplet corresponds to 2 HRCA droplet fused with 1 IVT droplet.

### DETAILED DESCRIPTION OF THE INVENTION

The present invention provides a highly sensitive method for detecting or quantifying a truncating mutation in a target gene. This method allows for the analysis of a large number of individual target genes for truncating mutations in micro-droplets and for detection of mutations in a target gene at the 0.1% level. The method of the invention thus constitutes a unique approach to perform gene mutation analyses, particularly for clinical diagnostics. The invention is of interest for detecting truncating mutations in a gene with high sensitivity.

The present invention is based on the protein truncation test (PTT). PTT is a technique for the detection of nonsense and frameshift mutations which lead to the generation of truncated protein products. Typically, the PTT technique involves the incorporation of a promoter site, ribosome binding site, and an artificial methionine start codon into a PCR product covering the region of the gene to be investigated. The PCR product is then transcribed and translated using a cell-free translation system, such as rabbit reticulocyte lysate, wheat germ lysate or E. coli lysate, to generate a protein encoded by the region of the amplified gene. The presence of a premature stop codon in the sequence, generated by a nonsense mutation or a frameshift, results in the premature termination of protein translation, producing a truncated protein that can be detected by standard gel electrophoretic separation of radio labelled proteins with an autoradiographic readout.

In the prior art, PTT normally requires incorporation of a chemical label (i.e. S³⁵ methionine, fluorescenated lysine) into the nascent peptide and this chemical label gives a signal whether or not it is incorporated into the peptide, therefore the unincorporated label must be physically separated from the test proteins prior to detection. This is accomplished by a separation step, typically gel-electrophoresis, which also allow wild type peptide and mutant truncated peptides to be separated due to their different electrophoretic mobilities. Elisa-PTT avoids separation of the mutant truncated peptide from the wild type peptide by measuring the relative amounts of N and C terminal tags on nascent peptides captured on a surface. However, the chemically labelled antibodies that recognize the N and C termini must be washed away prior to detection of bound label, therefore a separation step involving the removal of unattached label is still required. Mass spectrometry can be used to detect the mutant and wild type peptides (WO 2008/08484), but a desalting purification step is required before MALDI-TOF can be performed and mass spectrometry is a separation step in itself.

The inventors identified that massively parallel in vitro translation can be performed in droplets using one PCR product per droplet (or a clone of PCR products amplified from a single template) and could provide for a highly sensitive assay capable of detecting very low levels of mutant DNA in a sample. However, no separation steps are possible with droplets. Substances can be added to droplets, but cannot be easily removed. Therefore, a PTT that does not require removal of unincorporated label is advantageous when using the droplet platform. A PTT done in a droplet should therefore generate a signal that does not require separation of unincorporated label from incorporated label. One way to do this is to tag the C terminus of the test peptide with a peptide sequence that generates a signal, either directly or indirectly, that is not present in the absence of the C terminal tag. The C terminal tag must actively do something. An epitope tag recognized by an antibody would not be sufficient, unless the epitope tag acted in some positive way on the antibody to generate a signal, for example a conformational change in the antibody. A droplet that contains the C terminal tag sequence will have the signal generated by the tag, and will be considered as having a wild type test sequence, while a droplet lacking the C terminus must have a truncating mutation in the test sequence and would be considered mutant. Examples of C terminal tags that could be used for a droplet PTT are fluorescent proteins (such as GFP) and enzymes that act on a substrate present in the droplet, resulting in a detectable change, such as a change in color or fluorescence, for example a protease that cleaves a FRET peptide substrate.

Note that since each droplet has only one PCR product, the signal will be either positive or negative and relative quantitation of the signal in the droplet is not required.

A negative signal in a droplet could mean that the test sequence is mutant, or that it is wild type but was not translated, or that no template is present in the droplet. However, the presence of the translated test sequence in the droplet can be inferred by a number of means, such as by tagging the N terminus of the test sequence with a tag that generates a signal different from the C terminal tag, or by using a PCR product that encodes a polycistronic mRNA. In addition to the test sequence with or without the C terminal tag, the polycistronic mRNA could code, downstream of the test sequence, for a second peptide that gives a constitutive signal (different from the C terminal tag on the test sequence) indicating the presence of the mRNA, and hence the test sequence, but also the successful translation of the mRNA. Another option would be to have the PCR product encode 2 different mRNAs, one with the test sequence and one with a marker for the presence of the PCR product in a functional droplet. Importantly, these second two option do not require tagging of the nascent protein itself.

### DEFINITIONS

As used herein, the term “truncating mutation” refers to a mutation in a DNA coding sequence which results in an abnormally shortened protein, i.e. a truncated protein. This type of mutation may be due to nonsense or frameshift mutation. A nonsense mutation is a single base alteration in a DNA sequence that changes a codon recognized by a tRNA to stop codon recognized by release factors. A frameshift mutation is a mutation caused by an insertion or deletion of any number of nucleotides which alter the reading frame. Since the two alternative frames for any coding sequence have frequent stop codons, the frameshift will almost always result in a truncated protein.

A “stop codon” is a nucleotide triplet within messenger RNA that signals a termination of translation. In the standard human genetic code, there are three stop codons: UAG (“amber”), UAA (“ochre”), and UGA (“opal” or “umber”). As used herein, a “premature stop codon” means the occurrence of a stop codon where a codon corresponding to an amino acid should be.

As used herein, the term “target gene” refers to the gene bearing or suspecting of bearing the truncating mutation which has to be detected or quantified. This target gene can be any type of gene. In one embodiment, the target gene is a tumor suppressor gene.

A “tumor suppressor gene” is a gene that protects a cell from uncontrolled growth. When a tumor suppressor gene is mutated to cause a loss or reduction in its function, the cell can progress to cancer. Examples of tumor suppressor genes are the Rb gene of retinoblastoma, p 53, WT1, APC, hMSH2, hMLH1, BRCA1, BRCA2, BR1P1, FOXC2, CBP, FAP, ACVR2, NHS, PTEN, NF1 and NF2.

As used herein, the term “test sequence” refers to a polynucleotide sequence which is comprised in the target gene. The test sequence may be a portion or the entirety of the target gene sequence. The test sequence may be chosen in a region of the target gene most frequently affected by mutations, in particular by truncating mutations. In a particular embodiment, the target gene is the APC gene and the test sequence comprises the Multiple Cluster Region (MCR) of the APC gene.

As used herein, the term “in vitro compartmentalization”, or IVC, refers to an emulsion based technology, originally developed for screening and directed evolution of proteins and RNAs, that generates cell-like compartments in vitro (Tawfik and Griffiths, 1998). These compartments are aqueous microdroplets of water-in-oil emulsions and are designed such that each contains no more than one genetic construct.

The term “genetic construct” refers to a nucleic acid fragment that encodes for expression of one or more specific proteins. As used herein, the genetic construct may be linear, circular, double-stranded or single-stranded.

The term “promoter” refers to a regulatory region of DNA generally located upstream of a gene and capable of controlling the expression of a coding sequence. Promoters may be derived in their entirety from a native gene, or be composed of different elements derived from different promoters found in nature, or even comprise synthetic DNA segments. Promoters should be chosen by the skilled person according to the in vitro transcription and translation system used in the present invention. Promoters which may be used in the present invention include, without limitation, prokaryotic promoters such as trp, lacI, lacZ, T3, T7, SP6, gpt, lambda PR and lambda PL promoters, promoters from operons encoding glycolytic enzymes such as 3-phosphoglycerate kinase (PGK) and the acid phosphatase promoter; and eukaryotic promoters such as the CMV immediate early promoter, the HSV thymidine kinase promoter, heat shock promoters, the early and late SV40 promoter and the mouse metallothionein-I promoter. Preferably, the promoter is the T7 or SP6 promoter.

The phrase “operably linked” as used herein, refers to a nucleic acid sequence placed into a functional relationship with another nucleic acid sequence. A promoter is operably linked to a coding sequence if the promoter affects the transcription or expression of the coding sequence. Generally, operably linked DNA sequences are contiguous and are covalently linked together.

As used herein, the term “ribosome binding site” or “RBS” refers to any region of an eukaryote or prokaryote mRNA that is recognized by the ribosome in order to initiate the translation. This sequence can differ from a native RBS (e.g., a RBS found in a naturally-occurring gene) by at least one nucleotide. In one embodiment, the ribosome binding site comprises a Kozak sequence (Kozak, 1984). In another embodiment, the ribosome binding site comprises a Shine-Dalgarno (SD) sequence.

A “reporter system”, as used herein, may be any system capable of generating a detectable signal indicative of the presence and/or the activity and/or the expression of one or more genetic constructs encoding polypeptides. A reporter system may comprise one or several reporter genes.

As used herein a “reporter gene” or a “marker gene” is a gene whose expression may be assayed, i.e. a nucleic acid encoding a product that is readily detectable such as a fluorescent or colored product and/or encoding a product exhibiting a detectable activity with or without substrate. Many such genes are known in the art. Reporter genes include, without limitation, those encoding glucuronidase (GUS), luciferase, β-Glucosidase, alkaline phosphatase, horseradish peroxidase, beta-galactosidase (LacZ), green fluorescent protein (GFP) and derivatives such as EGFP, blue fluorescent proteins (EBFP, EBFP2, Azurite, mKalama1), cyan fluorescent proteins (ECFP, Cerulean, CyPet) and yellow fluorescent proteins (YFP, Citrine, Venus, YPet), DsRed and derivatives thereof, and Keima and derivatives thereof.

An “affinity reporter system”, as used herein, is a reporter system based on two or more members capable of associating with each other, covalently or not. In the present invention, at least one of the members is able to generate a signal. Preferably, the affinity reporter system is based on a biotin tag and fluorescent beads with covalently-attached streptavidin or avidin. Another example is digoxygenin and anti-digoxygenin antibodies, with the antibodies attached to fluorescent beads. These detection systems are useful for detecting a DNA in the form of a PCR product. This is done by covalently incorporating biotin or digoxygenin into the primers used for PCR amplification.

The terms “upstream” and “downstream”, as used herein, refer to a position of a genetic element on a polynucleotide sequence in relation to another genetic element. A first genetic element is upstream to a second genetic element when located in the 5′ direction of the coding strand from said second element. A first genetic element is downstream to a second genetic element when located in the 3′ direction of the coding strand from said second element.

As used herein, coding nucleic acid sequences are “fused in frame” when their translation results in a single polypeptide, a fusion protein. These fusion proteins are created through the joining of two or more genes which originally coded for separate proteins. Coding nucleic acid sequences may be fused in frame directly, or indirectly, via a linker sequence or spacer.

A “polycistronic mRNA”, as used herein, is a mRNA comprising several protein coding regions and thus which are translated into several proteins. The translation of internal coding regions usually requires the presence of an internal ribosome binding site, such as a Shine-Delgarno sequence, (in prokaryotic systems) or an internal ribosome entry site (in eukaryotic systems) upstream to said region.

An “internal ribosome entry site” (IRES) is a cis-acting RNA sequence able to mediate internal entry of the ribosome on an mRNA upstream of a translation initiation codon. These sequences are very diverse and may be chosen by the skilled person according to the desired properties among identified IRESs in the database (Bonnal et al., 2003).

The term “to diagnose” refers to the ability to determine or identify whether an individual has a particular disorder (e.g., a condition, illness, disorder or disease). In the present invention, this particular disorder is induced or partially induced by a truncating mutation in the target gene. Alternatively, the truncating mutation can be a marker associated with a particular disorder and not a causative agent.

The term “to prognosticate” refers to the ability to predict the outcome or prognosis of a disease. For instance, this term may refers to the ability to detect a pre-cancerous disorder and thus to predict the formation of a tumor, the occurrence of metastasis or the relapse of a cancerous disorder.

When the present invention refers to a method for diagnosing or prognosticating a disorder, it is also intended that the present invention concerns a method for providing data useful for diagnosing or prognosticating a disorder.

Method for Detecting or Quantifying a Truncating Mutation in a Target Gene

The present invention provides a method for detecting or quantifying a truncating mutation of a target gene in a subject.

In a first aspect, the present invention provides a method comprising:
a) providing a DNA sample from the subject;
b) assembling genetic constructs, each construct comprising a test sequence of said target gene, obtained from said DNA sample, operably linked with a promoter and a ribosome binding site, a first reporter system used to control the presence of said test sequence in the construct and/or the presence of a construct comprising said test sequence in a droplet, and a second reporter system used to detect the presence of a truncating mutation of said target gene, said first and second reporter systems generating distinct signals;
c) compartmentalizing each genetic construct in a droplet by forming a water-in-oil emulsion;
d) transcription and translation of each genetic construct in each droplet
e) monitoring emitted signals from said first and second reporter systems in each droplet to detect or quantify truncating mutations of said target gene.

Each step of this method is detailed below.

Step a): Providing a DNA Sample from the Subject

The nucleic acid sample, preferably a DNA sample, is obtained from a subject tissue sample by genomic DNA isolation using commonly practiced methods. It can also be prepared by RNA isolation and/or cDNA preparation.

A variety of DNA sample sources are contemplated, including but not limited to, tissue samples from a biopsy, amniotic fluid, urine, blood and stool samples.

The choice of the subject sample depends on the location of the potential pre-cancerous growth in the body. The pre-cancerous growth sheds cells (and hence mutant DNA) and this DNA can be isolated from a tissue sample containing the shed cells. Tumors anywhere in the body shed DNA into circulating blood, therefore blood is a target tissue for all tumors.

In a preferred embodiment, the method of the invention is used to diagnose or to prognosticate a colorectal cancer and the DNA sample is obtained from a stool sample.

In an embodiment, the method of the invention further comprises an amplification step of the test sequence from the DNA sample before assembling genetic constructs. Amplification may be by any technique, including, but not limited to, QP-replicase amplification (Cahill et al., 1991; Chetverin and Spirin, 1995; Katanaev et al., 1995), the ligase chain reaction (LCR) (Landegren et al., 1988; Barany, 1991), the self-sustained sequence replication system (Fahy et al., 1991), strand displacement amplification (Walker et al., 1992), nucleic acid sequence-based amplification (NASBA) (Compton, 1991), loop-mediated isothermal amplification (Notomi et al., 2000), rolling circle amplification (RCA) (Blanco et al., 1989) and hyperbranched rolling circle amplification (HRCA) (Lizardi et al., 1998). Preferably amplification is by PCR (Saiki et al., 1988) or hyperbranched rolling circle amplification (HRCA).

The test sequence is chosen by the skilled person and may correspond to a portion of the target gene most frequently affected by mutations.

In a particular embodiment, the target gene is the APC gene and the test sequence comprises the Multiple Cluster Region (MCR) of the APC gene (codons 1286-1513 of the APC gene; GenBank Accession number M74088).

In this embodiment, the sensitivity of the method of the invention depends in part on the error rate of the polymerase. Thus, a high-fidelity PCR polymerase should be used in order to limit mutations introduced by the error rate intrinsic to PCR. Preferably the error rate of the polymerase is less than 10⁻⁵, more preferably less than 10⁻⁶, and the most preferably less than 5.10⁻⁷ (for example, Phusion Polymerase from New England Biolabs exhibiting an error rate of 4.4×10⁻⁷).

For the PCR amplification, a set of primers are needed. Suitable primers comprise a region complementary to the template and can be designed by the skilled person based on the known nucleic acid sequence of the target gene. This sequence can be found, for instance, in nucleic acid sequence databases such as GenBank.

A set of primers comprise at least two primers, a forward primer and a reverse primer.

In an embodiment, the forward primer contains a start codon used for the translation of the test sequence. In another embodiment, the forward primer is complementary of the target gene just upstream of its naturally occurring start codon and this start codon is used for the translation of the test sequence.

In one embodiment, the forward primer further comprises, on its 5′-end portion, a promoter and a ribosome binding site, to be both operably linked with the test sequence of the target gene. Optionally, the forward primer can also have an additional promoter, preferably at its extreme 5′ end, in a reverse orientation to be operably linked with a marker gene upstream to the test sequence and in a reverse orientation compared with the test sequence in the genetic construct.

In another embodiment, the forward primer comprises a ribosome binding site to be operably linked with the test sequence and a promoter in a reverse orientation. In this embodiment, the promoter will be operably linked in the further genetic construct with a marker gene upstream to the test sequence and in a reverse orientation compared with test sequence. The further genetic construct will contain the promoter for the test sequence. Optionally, the forward primer can further comprise an additional promoter to be operably linked with the test sequence of the target gene.

The forward primer and/or the reverse primer may comprise recognition sites of restriction enzymes. Preferably, these restriction enzymes do not cut within the test sequence.

It should be taken care that the test sequence and the reverse primer do not comprise any stop codon in the correct reading frame.

Accordingly, the resulting test sequence of the target gene comprises at least a start codon and the coding sequence of the target gene or a part thereof. In addition, the resulting test sequence can further comprise a promoter and a ribosome binding site operably linked to the coding sequence of the target gene. Alternatively, the resulting test sequence can further comprise a promoter upstream to the coding sequence of the target gene and in a reverse orientation, and a ribosome binding site operably linked to the coding sequence of the target gene. In a further embodiment, the resulting test sequence of the target gene comprises a promoter and a ribosome binding site operably linked to the coding sequence of the target gene and a promoter upstream to the coding sequence of the target gene and in a reverse orientation.

Optionally, the resulting test sequence of the target gene can comprise at its ends appropriate restriction enzymes recognition sites for assembly into genetic constructs.

Step b): Assembling Genetic Constructs

Genetic constructs containing a test sequence obtained directly from the DNA sample or after amplification are then assembled. Each construct comprises (i) a test sequence operably linked with a promoter and a ribosome binding site, (ii) a first reporter system used to control the presence of said test sequence in the construct and/or the presence of a construct comprising said test sequence in a droplet, and (iii) a second reporter system used to detect the presence of a truncating mutation of said target gene, said first and second reporter systems generating distinct signals.

In each genetic construct, the test sequence is operably linked with a promoter and a ribosome binding site. As detailed above, the test sequence obtained by amplification comprises a promoter operably linked with the test sequence and/or a promoter in a reverse orientation and upstream to the test sequence. If the test sequence does not comprise any promoter operably linked with the test sequence, such a promoter has to be provided when genetic constructs are assembled.

Each genetic construct comprises a second reporter system used to detect the presence of a truncating mutation in the target gene.

In an embodiment, the second reporter system is a marker gene operably linked to the test sequence in order to be expressed in a single mRNA, the marker gene being downstream to the test sequence.

In a particular embodiment, the second reporter system is a marker gene fused in frame with the test sequence. In this embodiment, an overlap PCR can be performed to fuse the test sequence in frame with a marker gene. However, any known method allowing the in frame fusion of the test sequence with the marker gene is contemplated by the present invention, for instance by using appropriate restriction enzymes digests and by ligation.

In a preferred embodiment, the second reporter system is the LacZ gene fused in frame with the test sequence of the target gene.

Each genetic construct comprises a first reporter system used to control the presence of the test sequence in the construct and/or the presence of a construct comprising the test sequence in a droplet.

In a first embodiment, the first reporter system is a marker gene expressed from a promoter which is operably linked with said marker gene only if the test sequence of the target gene is present in the construct.

In a particular embodiment, the test sequence comprises a promoter in a reverse orientation. This promoter, which may be appended by amplification, is then operably linked with the marker gene of the first reporter system when the genetic construct is assembled. In this configuration, the marker gene of the first reporter system is in reverse orientation to the test sequence. Moreover, a ribosome binding site is placed between the marker gene of the first reporter system and its promoter.

In order to assemble the genetic construct according to this embodiment, an overlap PCR may be performed to combine the test sequence, the second reporter system and the marker gene of the first reporter system. This construct can be assembled in two steps, first the test sequence is fused to the first reporter gene and then the second, or vice versa. However, any known method allowing assembly of the genetic construct is contemplated by the present invention, for instance by using appropriate restriction enzymes digests and by ligation.

The DNA fragment carrying the marker gene of the first reporter system further comprises a ribosome binding site for the translation of this marker gene and optionally a promoter in a reverse orientation to said marker gene which is operably linked to the test sequence when assembled in the construct if the test sequence does not already contain such a promoter. This embodiment is exemplified in the experimental section (example 3).

In a preferred embodiment, the resulting construct comprises:
in one orientation and operably linked, a promoter, a ribosome binding site and a test sequence of the target gene fused in frame with the second marker gene; and,
in a reverse orientation and operably linked, a promoter, a ribosome binding site and the first marker gene.

In another embodiment, the first reporter system is a marker gene expressed on a polycistronic mRNA further comprising the test sequence fused in frame with the marker gene of the second reporter system and an internal ribosome entry site or an internal ribosome binding site, said test sequence being downstream to the first reporter system and upstream to the second reporter system and said internal ribosome entry site or internal ribosome binding site being operably linked to the test sequence fused in frame with the marker gene of the second reporter system.

When the first reporter system is a marker gene, genetic constructs may also be assembled by using a circular vector such as a plasmid, as exemplified in the experimental section (example 1). In this case, the test sequence has restriction sites at both ends and is directionally ligated into the vector following digestion with restriction enzymes. Restriction enzymes have to be chosen to not cut within the test sequence. The vector contains marker genes of the first and the second reporter systems. The test sequence is properly placed into the vector in order to allow expression of the first reporter system using a monocistronic message and of a second monocistronic mRNA comprising the coding sequence of the target gene and the marker gene of the second reporter system, preferably fused in frame.

In this embodiment, there can be no expression of the test sequence of the target gene and the second reporter system, in the absence of the marker gene of the first reporter system in the genetic construct. In addition, there can be no expression of the first reporter system marker gene in the absence of the test sequence.

In a preferred embodiment, the marker genes of the first and second reporter systems encode distinct proteins. These proteins include, without any limitation, beta-galactosidase, beta-glucuronidase, beta-glucosidase, luciferase, horseradish peroxidase, alkaline phosphatase, green fluorescent protein, DsRed, Keima and derivatives thereof.

In a particular embodiment, the first and the second reporter systems are marker genes and genetic constructs are amplified before compartmentalization. This amplification may be performed by any known technique such as described above and suitable primers will be easily chosen by the person skilled in the art.

In another embodiment, the first reporter system is an affinity reporter system comprising two members, a first member appended on the 5′ end of the coding strand of the test sequence and a second member able to bind the first member. The signal emitted from this reporter system is generated by the second member.

In a preferred embodiment, the first member of the affinity reporter system is a biotin tag and the second member a fluorescent streptavidin or avidin coated bead. In this embodiment, the primers used in the overlap PCR amplification to combine the test sequence and the second reporter system, append a biotin tag on the 5′ end of the coding strand of the test sequence. This construct is then mixed with fluorescent streptavidin or avidin coated beads. Alternatively, a primer of the first amplification comprises a biotin tag. Each genetic construct tagged with the first member of the first reporter system, i.e. the biotin tag, is thus bound to the second member of the first reporter system, i.e. a fluorescent streptavidin or avidin coated bead, which is able to generate a signal. This embodiment is exemplified in the experimental section (example 2).

Accordingly, in a particular embodiment, the resulting construct comprises:
a promoter, a ribosome binding site, both operably linked to a test sequence of the target gene fused in frame with the second marker gene; and,
a first member appended on the 5′ end of the coding strand of the construct.

In a particular embodiment, the method of the invention further comprises an additional step after assembling genetic constructs and before compartmentalizing in droplets in order to remove second members of the first reporter system which are not bound to the first member. Preferably this step is performed by affinity purification using a tag on the 5′ end of the non-coding strand of the genetic construct.

In a preferred embodiment, a digoxigenin tag is appended on the 5′ end of the non-coding strand of the product of the overlap PCR, i.e. downstream to the marker gene of the second reporter system, by reverse primers used in the overlap PCR. In this case, second members of the first reporter system with no DNA attached (which would give rise to false positives) are removed by affinity second members with a gene attached using, for example, commercially available non-fluorescent magnetic beads coated with an anti-Digoxigenin antibody, as exemplified in the experimental section (example 2).

Step c): Compartmentalizing Each Genetic Construct

Each genetic construct of step b) is then compartmentalized in a droplet, preferably by forming a water-in-oil emulsion.

A wide variety of microencapsulation procedures are available and may be used to compartmentalize each genetic construct in droplets in accordance with the present invention (Benita, 1996). Preferably, the droplets of the present invention are formed by emulsions. These emulsions are heterogeneous systems of two immiscible liquid phases with one of the phases dispersed in the other as droplets of microscopic or colloidal size (Becher, 1957; Sherman, 1968; Lissant, 1984). Emulsions may be produced from any suitable combination of immiscible liquids.

In a preferred embodiment, the emulsion of the present invention has water (containing the biochemical components) as the phase present in the form of finely divided droplets and a hydrophobic, immiscible liquid (an oil) as the matrix in which these droplets are suspended. Such emulsions are termed “water-in-oil”. The emulsion may be stabilised by addition of one or more surfactants.

Creation of an emulsion generally requires the application of mechanical energy to force the phases together. There are a variety of ways of doing this which utilise a variety of mechanical devices, including stirrers (such as magnetic stir-bars, propellers and turbine stirrers, paddle devises and whisks), homogenisers (including rotor-stator homogenisers, high-pressure valve homogenisers and jet homogenisers), colloid mills, ultrasound and ‘membrane emulsification’ devices (Becher, 1957; Dickinson, 1994). More recently, microfluidic emulsification techniques which allow for the creation of highly monodisperse emulsion have been developed, based on, for example, drop-breakoff in co-flowing streams, cross-flowing streams in a T-shaped junction, and hydrodynamic flow-focussing (reviewed by Christopher and Anna, 2007).

In an embodiment, genetic constructs are compartmentalized together with an in vitro transcription and translation system. Many suitable in vitro transcription and translation systems which will allow coupled transcription/translation are commercially available. Such systems typically combine a prokaryotic phage RNA polymerase and promoter (e.g. T7, T3, or SP6) with eukaryotic (e.g. rabbit reticulocyte or wheat germ) or prokaryotic (e.g. E. coli) extracts, or cell-free translation systems reconstituted with purified components (Shimizu et al., 2001), to synthesize proteins from DNA templates. The appropriate system may vary depending on the precise nature of the requirements in each application of the invention, as will be apparent to the skilled person.

In addition, genetic constructs can be compartmentalized together with one or several substrates necessary to generate reporter system signals. Preferably, these substrates give rise to fluorescent, luminescent or colored products. The appropriate substrate(s) may vary depending on the reporter systems used, as will be apparent to the skilled person. These substrates include, without limitation, 5-bromo-4-chloro-3-indolyl glucuronide (X-Gluc), p-nitrophenyl β-D-glucuronide, Resorufin β-D-glucuronide (RUG), 4-methylumbelliferyl-beta-D-glucuronide (MUG) and carboxyumbelliferyl β-D-Glucuronide (CUGlcU), if one reporter system comprises the GUS reporter gene; D-Luciferin, 6-Amino-D-luciferin and D-Luciferin-6-0-β-D-Galactopyranoside if one reporter system comprises a luciferase encoding gene; fluorescein-di-beta-D-galactopyranoside (FDG), fluorescein mono-β-D-Galactopyranoside (FMGa1), resorufin β-D-Galactopyranoside (Res-Gal), 4-Methylumbelliferyl β-D-Galactopyranoside, 4-Trifluoromethylumbelliferyl-β-D-Galactopyranoside (TFMU-Gal), D-Luciferin-6-0-β-D-Galactopyranoside and 2′,7′-Dichlorofluorescein di-β-D-galactopyranoside (DCFDG) if one reporter system comprises a beta-galactosidase encoding gene; Fluorescein diphosphate tetraammonium salt, 3-Phenylumbelliferone 7-O-phosphate hemipyridinium salt and resorufin-7-O-phosphate diammonium salt if one reporter system comprises an alkaline phosphatase encoding gene; Fluorescein mono-β-D-Glucopyranoside (FMGlc), resorufin β-D-Glucopyranoside and fluorescein di-β-D-Glucopyranoside (FDGlu) if one reporter system comprises a beta-glucosidase encoding gene. If several substrates have to be used, they should be chosen to be compatible with each other in an in vitro transcription and translation environment. Importantly, the in vitro translation system (which is usually a cell lysate) must have low endogenous activity of the reporter enzymes being used.

In a preferred embodiment, reporter systems comprise the LacZ and GUS genes with fluorescein-di-beta-D-galactopyranoside and resorufin β-D-glucuronide as substrates, respectively, and are added to the mix comprising genetic constructs and a rabbit reticulocyte lysate in vitro transcription and translation system, which has very low levels of endogenous GUS and LacZ activity.

In a typical in vitro compartmentalization process, a 50 μl of mix comprising in vitro transcription and translation system, genetic constructs and optionally reporter system substrates, is dispersed into ˜10¹⁰ droplets, in a water-in-oil emulsion. The DNA concentration is chosen such that ˜10% of these droplets contain a single genetic construct (the rest containing no genetic constructs), and very few droplets contain more than one genetic construct.

In an embodiment, genetic constructs are amplified after compartmentalization of step c) and the method further comprises, after said amplification, an additional step of fusing droplets containing said amplified genetic constructs with droplets containing an in vitro transcription and translation system. Amplification within droplets and droplet fusion are performed as described in the second aspect of the invention.

Step d) Transcription and Translation of Genetic Constructs in Droplets

After compartmentalization, conditions are set in order to allow in vitro transcription and translation of each genetic construct in each droplet.

These conditions may be set by the skilled person or according to the recommendations of the transcription and translation system manufacturer.

Step e): Monitoring Emitted Signals from Reporter Systems

After compartmentalization, the activities of the two reporter systems in each droplet are monitored simultaneously thanks to distinct emitted signals. The presence of the first reporter signal indicates that the droplet has everything needed to generate the second reporter signal, thus the absence of the second reporter signal is meaningful and indicates the presence of a truncating mutation in the test sequence.

In a preferred embodiment, reporter systems are chosen in order to generate fluorescent signals and emitted signals from each droplet are monitored using epifluorescence microscopy and scored using image analysis software.

All droplets containing a properly made construct contain the test sequence of the target gene (mutated or unmutated) and emit a signal generated by the first reporter system. Droplets that do not emit a signal from the first reporter are not taken into account. The lack of the first reporter signal can be due to the absence of a DNA construct, the presence of an inhibitor of translation, or the absence of a component needed for in vitro translation.

When the test sequence of the target gene does not comprise a truncating mutation, the droplet emits not only the signal generated by the first reporter system but also the signal generated by the second reporter system.

When the test sequence of the target gene comprises a truncating mutation, the droplet only emits the signal generated by the first reporter system.

The ratio of droplets emitting signals generated by the two reporter systems to droplets emitting only the signal generated by the first reporter allows quantification of the truncating mutation in the target gene of the tissue sample from the subject.

In an embodiment, the method of the invention further comprises an additional step of sorting the droplets after monitoring emitted signals from reporter systems.

Target genes containing truncating mutations can be recovered to allow further characterization or manipulation (for example, sequencing) by sorting droplets exhibiting the appropriate reporter signals and breaking the recovered emulsion.

The fluorescence of droplets can be analysed and the droplets sorted at high speeds using fluorescence-activated cell sorting (FACS) (Eisenstein, 2006). For example, water-in-oil-in-water double emulsions have previously been used to sort genes based on the activity of the enzymes they encode (via in vitro transcription-translation) using FACS (Mastrobattista et al., 2005). Alternatively, microfluidic flow sorting systems could be used, including, but not limited to; systems that exploit electrokinetic actuation (Li and Harrison, 1997; Fu et al., 1999; Dittrich and Schwille, 2003), optical forces (Wang et al., 2005; Perroud et al., 2008), hydrodynamic flow-switching (Fu et al., 2002; Kruger et al., 2002; Wolff et al., 2003; Ho et al., 2005), or dielectrophoretic actuation (Lapizco-Encinas et al., 2004; Hu et al., 2005; Kim et al., 2007). Preferably, droplet sorting would be preformed using dielectrophoretic droplet actuation (Ahn et al., 2006). Sorted emulsions can be broken either using chemical emulsion breakers (Clausell-Tormos et al., 2008), or using electrocoalescence (Fidalgo et al., 2008).

In a second aspect, the present invention provides a method comprising:
a) providing a DNA sample from the subject;
b) compartmentalizing each DNA molecule, from said DNA sample, comprising a test sequence of said target gene in first droplets by forming a water-in-oil emulsion;
c) assembling genetic constructs, each construct comprising said test sequence operably linked with a promoter and a ribosome binding site, a marker gene of a first reporter system used to control the presence of said test sequence in the construct and/or the presence of a construct comprising said test sequence in a droplet, and a marker gene of a second reporter system used to detect the presence of a truncating mutation of said target gene, said first and second reporter systems generating distinct signals;
d) fusing first droplets containing genetic constructs of step c) with second droplets containing an in vitro transcription and translation system;
e) transcription and translation of each genetic construct in each fusion droplet
f) monitoring emitted signals from said first and second reporter systems in each fusion droplet to detect or quantify truncating mutations of said target gene.

Steps of providing a DNA sample from the subject (a), transcription and translation of each genetic construct (e) and monitoring emitted signals from reporter systems (f) are performed as described above.

In an embodiment, the method further comprises an amplification step of the test sequence from the DNA sample before step b).

This amplification may be performed as described in the first aspect of the invention, in particular by using the polymerase chain reaction or Hyperbranched Rolling Circle Amplification.

If amplification is performed by using HRCA, DNA sample has to be circularised, for example by ligation, before amplification.

In this aspect, each DNA molecule, obtained from the DNA sample, directly or after an amplification step, is compartmentalized in first droplets by forming a water-in-oil emulsion. This compartmentalization is performed as described above.

In an embodiment, the method further comprises an amplification step of the test sequence from DNA molecules contained within droplets after step b) and before step c).

Amplification within Droplets

In order to perform amplification within droplets, DNA molecules are compartmentalized together with amplification reagents necessary to assemble genetic constructs within droplets and, optionally to amplify the test sequence and/or genetic construct within droplets.

The amplification may be performed by any known technique, preferably by PCR or HRCA. Examples of PCR or HRCA performed within droplets are presented in the experimental section (examples 4 and 5).

For PCR amplification the droplets need to be stable to thermocycling, whereas this is not required for the isothermal HRCA technique. PCR amplification of single DNA (or RNA) molecules compartmentalized in emulsion droplets (emulsion PCR) is widely practiced and has a range of applications (reviewed in Kelly et al., 2007).

The genetic constructs comprising marker genes of first and second reporter systems are assembled by amplification within droplets as described above. For example, the genetic constructs can be assembled using overlap PCR, as previously exemplified to amplify and link rearranged immunoglobulin heavy and light chain V-genes compartmentalized within single cells (Embleton et al., 1992).

In an embodiment, the second reporter system is a marker gene operably linked to the test sequence in order to be expressed in a single mRNA, said marker gene being downstream to said test sequence. Preferably, the marker gene of the second reporter system is fused in frame with the test sequence.

In a particular embodiment, the marker gene of the first reporter system is expressed on a polycistronic mRNA further comprising the test sequence fused in frame with the marker gene of the second reporter system and an internal ribosome entry site or an internal ribosome binding site, said test sequence being downstream to said first reporter system and upstream to said reporter system, and said internal ribosome entry site or internal ribosome binding site being operably linked to said test sequence fused in frame with said marker gene of said second reporter system

In another embodiment, the marker gene of the first reporter system is expressed from a promoter which is operably linked with said marker gene only if said test sequence is present in the construct.

In an embodiment, marker genes of first and second reporter systems are different and are selected from the group consisting of beta-galactosidase, beta-glucuronidase, beta-glucosidase, luciferase, horseradish peroxidase, alkaline phosphatase, green fluorescent protein, DsRed, Keima and derivatives thereof.

In a preferred embodiment, the marker gene of the first reporter system is the beta-glucuronidase encoding gene and the marker gene of the second reporter system is the beta-galactosidase encoding gene.

Suitable primers and amplification procedures used in all of these embodiments have been detailed in the first aspect of the invention.

In a particular embodiment, assembled genetic constructs are amplified before the droplets are fused with droplets containing in vitro transcription and translation system. The choice of suitable primers will be obvious for the skilled person.

Droplet Fusion

In order to perform transcription and translation of the genetic constructs, first droplets containing these constructs have to be fused with second droplets containing an in vitro transcription and translation system. This fusion may be performed by any technique known by the skilled person.

In a particular embodiment, the fusion is performed by using a droplet fusion device. The droplet fusion device consist of four separate modules integrated into single microfluidic chip, that is, (i) droplet reinjection, (ii) on-chip droplets generation, (iii) droplets pairing and (iv) electro-coalescence modules. The depth of all channels is 20 μm.

On-chip droplet generation module has a T-shaped junction with 10 μm wide and 15 μm long constriction. Channel down the nozzle is 30 μm wide and 3.5 mm long. The size of the droplet is controlled by adjusting the flow rates of aqueous phase and carrier oil using syringe pumps.

Droplet reinjection module consists of Ψ-shaped structure where droplets are spaced by carrier oil such as FC40 fluorinated liquid (3M®) with REA 3% (w/w). To increase the spacing between reinjected droplets 10 μm wide channel is used just before merging a pairing channel.

A pair of droplets is formed in the pairing channel of 20 μm wide and 800 μm long, just before electro-coalescence region. Noteworthy, in channels longer than 1 mm droplets tend to form a group of double or triple pairs thereby increasing the number of non-desirable fusion events.

Electro-coalescence region contains a channel with the turn, where two electrodes are placed perpendicular to the flow direction. An electric field is generated by applying 600 mV ac at 30 kHz across electrodes spaced 120 μm.

Finally, after electro-coalescence droplets flow in the 5.5 mm long exit channel before entering the collection outlet. Shielding electrodes are used in order to prevent non-desirable droplets electro-coalescence during emulsion reinjection and collection.

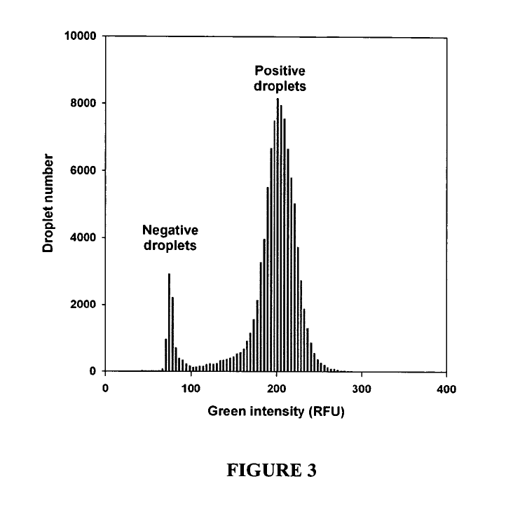

FIG. 3 is a schematic representation of the droplet fusion device as described above. Using this device, droplets containing in vitro transcription and translation system are generated and fused with reinjected droplets of step c) containing genetic constructs.

In an embodiment, the first and/or second droplets further contain one or several substrates necessary to generate reporter system signals.

In an embodiment, the method further comprises an additional step after step f) of sorting the droplets to allow further characterisation or manipulation of the test sequence. This sorting may be performed as described above.

Genetic Constructs

In a third aspect, the present invention provides genetic constructs which may be used according to the invention.

In an embodiment, the genetic construct comprises a test sequence operably linked with a promoter and a ribosome binding site, a first marker gene operably linked with another promoter, and a second marker gene which is operably linked to the test sequence in order to be expressed in a single mRNA, said marker gene being downstream to said test sequence. Preferably, the test sequence comprises all or part of the APC gene.

In a particular embodiment, the first and second marker genes are different and are selected from the group consisting of genes encoding beta-galactosidase, beta-glucuronidase, beta-glucosidase, luciferase, horseradish peroxidase, alkaline phosphatase, green fluorescent protein, DsRed, Keima and derivatives thereof.

In a preferred embodiment, the first marker gene is the beta-glucuronidase encoding gene and the second marker is the beta-galactosidase encoding gene.

In another embodiment, the genetic construct comprising a test sequence operably linked with a promoter and a ribosome binding site, a first reporter system which is an affinity system comprising two members, a first member of the first reporter system appended on the 5′ end of the coding strand of the test sequence and a second member of the first reporter system which is bound to the first member and is able to generate a signal, and a marker gene which is operably linked to the test sequence in order to be expressed in a single mRNA, said marker gene being downstream to said test sequence. Preferably, the test sequence comprises all or part of the APC gene.

In a particular embodiment, the first member of the affinity system is a biotin tag and the second member is a fluorescent steptavidin coated bead.

In another particular embodiment, the marker gene is selected from the group consisting of genes encoding beta-galactosidase, beta-glucuronidase, beta-glucosidase, luciferase, horseradish peroxidase, alkaline phosphatase, green fluorescent protein, DsRed, Keima and derivatives thereof. Preferably, the marker gene is the beta-galactosidase encoding gene.

In another aspect, the present invention provides a droplet from a water-in-oil emulsion containing a genetic construct comprising at least two reporter systems generating distinct signals.

The droplet is obtained from a water-in-oil emulsion as described above.

The genetic construct comprises in the droplet may be one of the present invention or any other genetic constructs comprising at least two reporter systems generating distinct signals.

Diagnosis and Prognosis

The method according to the invention may be used to diagnose or to prognosticate any disease related to a nonsense or frameshift mutation which results in a truncated gene product, i.e. both the existence of disease and the predisposition to disease may be tested. These diseases include, without limitation, colorectal cancer (Traverso et al., 2002), breast and ovarian cancer (Garvin et al., 1998), polycystic kidney disease (Peral et al., 1997), neurofibromatosis (Hein et al., 1995) and Duchenne muscular dystrophy (Roest et al., 1993).

In a particular embodiment, the disease is a colorectal cancer and the target gene is the APC gene.

In another embodiment, the disease is a breast cancer or an ovarian cancer and the target gene is the BRCA1 or BRCA2 gene.

In another embodiment, the disease is a polycystic kidney disease and the target gene is the PKD1 gene.

In another embodiment, the disease is a neurofibromatosis and the target gene is the HF1 or NF2 gene.

In another embodiment, the disease is a Duchenne muscular dystrophy and the target gene is the DMD gene.

The method according to the invention may be used in to diagnose or to prognosticate a disease in any subject in need thereof. This subject is preferably a mammal, more preferably a human. Humans of all ages can be tested and the present invention contemplates also pre-natal tests (e.g. by using fetal DNA in maternal blood as sample). The high sensitivity of the method of the invention allows use of the maternal blood as sample which has only trace amounts of fetal DNA instead of amniotic fluid and thus allows non-invasive pre-natal tests.

Kits

The present invention also relates to a kit for the detection or quantification of a truncating mutation in a target gene in a subject by using the method according to the invention. The kit of the invention comprises at a minimum reagents needed to assemble genetic constructs of the invention, an in vitro transcription/translation system, reagents needed to form a water-in-oil emulsion and means needed to compartmentalize each genetic construct or DNA molecule into a droplet.

In an embodiment, the kit comprises primers suitable for amplifying the test sequence of the target gene and/or primers suitable for assembling the genetic construct.

In another embodiment, the kit comprises reagents needed to assemble genetic constructs of the invention, said reagents comprising a plasmid containing the marker gene of the first reporter system and/or the marker gene of the second reporter system.

In another embodiment, the kit further comprises one or more substrates needed to generate reporter system signals.

In another embodiment the kit further comprises one or more surfactants. Preferably, the one or more surfactants are chosen from the group consisting of Span-80, Triton X-100 and ABIL EM 90.

In a preferred embodiment the kit contains mineral oil, ABIL EM 90 and rabbit reticulocyte lysate as the ingredients for making droplets that allow in vitro translation with a low background of the LacZ and GUS reporter genes, and the FDG and RUG substrates for LacZ and GUS.

The following examples are given for purposes of illustration and not by way of limitation.

### EXAMPLES

### Example 1

### Proof of Concept Using Plasmid Constructs

Wild type human genomic DNA or DNA from the SW480 cell line having a CAG to TAG truncating mutation at codon 1338 (bp 4075) in the MCR of the APC gene was used as template to amplify the 1.2 Kb MCR of the APC gene. The PCR product had a NotI restriction site at the 5′ end and an XmaI restriction site at the 3′ end. The PCR product was digested with these enzymes and purified before being directionally ligated into a plasmid vector, which has also been digested with NotI and XmaI. These enzymes do not cut within the APC test sequence as presented in the database. The T7 promoter in the opposite orientation allows expression of the reporter gene of the first reporter system (the GUS gene) when the PCR product is properly placed in the vector. The vector was placed in a 10 microliter volume capable of performing coupled in vitro transcription/translation. The vector also contains a reporter gene of the second reporter system (the LacZ gene) which is fused to the APC test sequence in frame. mRNA from the reporter gene of the second reporter system is expressed from the T7 promoter directed towards the amplified polynucleotide sequence and the enzyme is translated and expressed when the test sequence is wildtype, while no LacZ enzyme is expressed when the test sequence has a mutation that results in premature truncation of the protein product. Since virtually all clinically important mutations in the APC gene are the result of a premature truncation, this strategy detects the clinically important mutations.

The presence of the GUS enzyme activity and the LacZ enzyme activity were monitored using fluorescent substrates for these 2 enzymes. GUS cleaves Resorufin β-D-glucuronide (RUG) to yield Resorufin (excitation 525, emission 610), while LacZ cleaves fluorescein-di-D-galactopyranoside (FDG) to yield fluorescein (excitation 470, emission 525).

Results obtained after in vitro transcription/translation in the presence of enzyme substrates are presented in the table 1 below.

TABLE 1
quantification of the fluorescence obtained from bulk solution

| Bulk solutions | 525 nm emission (RFU) |
|---|---|
| No vector | 3 |
| Empty P3 vector | 4 |
| Vector having a wild type APC test sequence | 441 |
| Vector having an APC mutated test sequence | 6 |

LacZ was expressed when the APC test sequence comprised in the vector was wild type. Whereas, LacZ was not expressed when the APC test sequence comprised in the vector was a mutated sequence comprising a truncating mutation.

These results demonstrate that the method of the invention allows to efficiently detect truncating mutations in a gene.

### Example 2

### Detection of Polyps in Stool DNA by Using Genetic Constructs Comprising an Affinity Reporter System as First Reporter System

Genomic DNA is isolated from a stool sample using existing methodologies and the 1.2 Kb MCR of the APC gene is amplified using high-fidelity PCR (using Phusion Polymerase from New England Biolabs exhibiting an error rate of 4.4×10⁻⁷). This high-fidelity enzyme minimizes the number of false positives due to mutations introduced by PCR. An overlap PCR is then performed to fuse the APC gene in frame with the marker gene lacZ. The primers used to amplify the APC-lacZ fusion append biotin at one end (5′ end) of the construct and digoxigenin (DIG) at the other end (3′ end).

The products of the overlap PCR are then attached to commercially available streptavidin-coated red-fluorescent beads via the biotin tag by adding a molar excess of red-fluorescent beads.

Beads with no DNA attached (which would give rise to false positives) are removed by affinity purifying red-fluorescent beads with an overlap PCR product attached using commercially available non-fluorescent magnetic beads coated with an anti-Digoxigenin antibody.

The beads with overlap PCR products attached are then mixed with a commercial coupled in vitro transcription and translation system (on ice to prevent the reaction from starting) and fluorescein di-beta-D-galactoside (FDG) as substrate for the beta-galactosidase activity, and dispersed to form a water-in-oil emulsion. The concentration of beads is set such that very few droplets will contain more than one bead (or more than one overlap PCR product). The single overlap PCR product in each droplet is transcribed and translated. When the APC gene is unmutated, an APC-LacZ fusion protein is created. When the APC gene is mutated, only a truncated APC gene is produced, LacZ is not translated and there is no beta-galactosidase activity.

To score the droplets, the droplets are spread to form a monolayer on a slide, and analysed using epifluorecence microscopy. Droplets that do not contain a red-fluorescent bead with an overlap PCR product attached are non-fluorescent and are not scored. Droplets which contain an unmutated APC gene exhibit red-fluorescence due to the attached bead, and green fluorescence due to the enzymatic hydrolysis of FDG to fluorescein catalysed by the APC-LacZ fusion.

Droplets which contain mutated APC genes only exhibit red fluorescence due to the red-fluorescent bead. The ratio of red and green drops, to drops which are red alone gives the percentage of mutated APC genes.

Well over a million drops (each ˜10 μm in diameter) can be spread in monolayer of area less than 1 cm² on a slide. The presence of 0.1% mutated APC genes thus result in 10³ red and green drops out of 10⁶ red drops. With the method of the invention, the sensitivity of 0.1% is thus easily achieved.

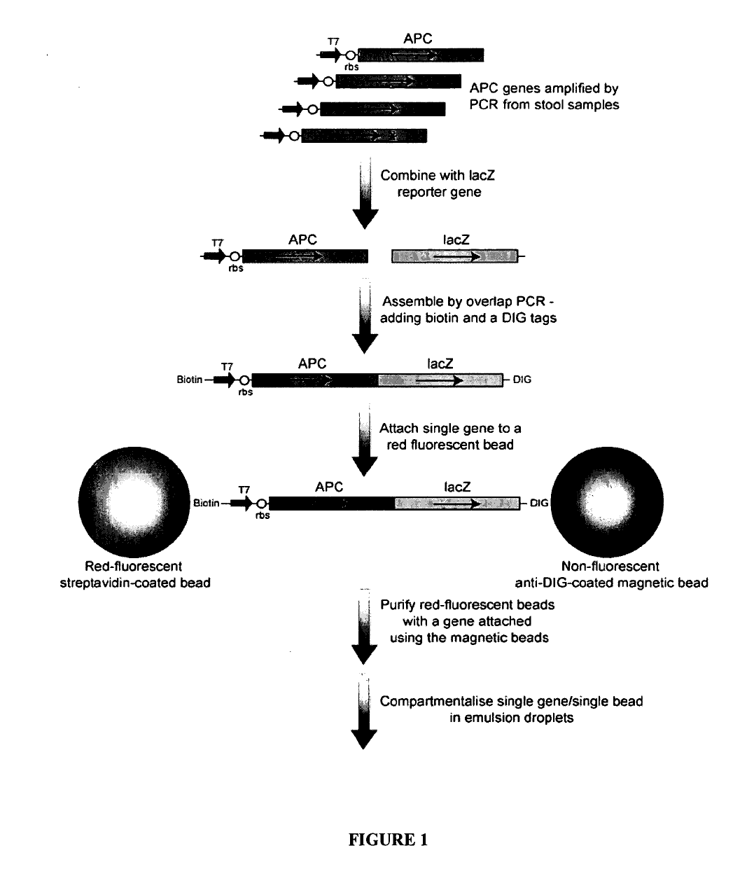

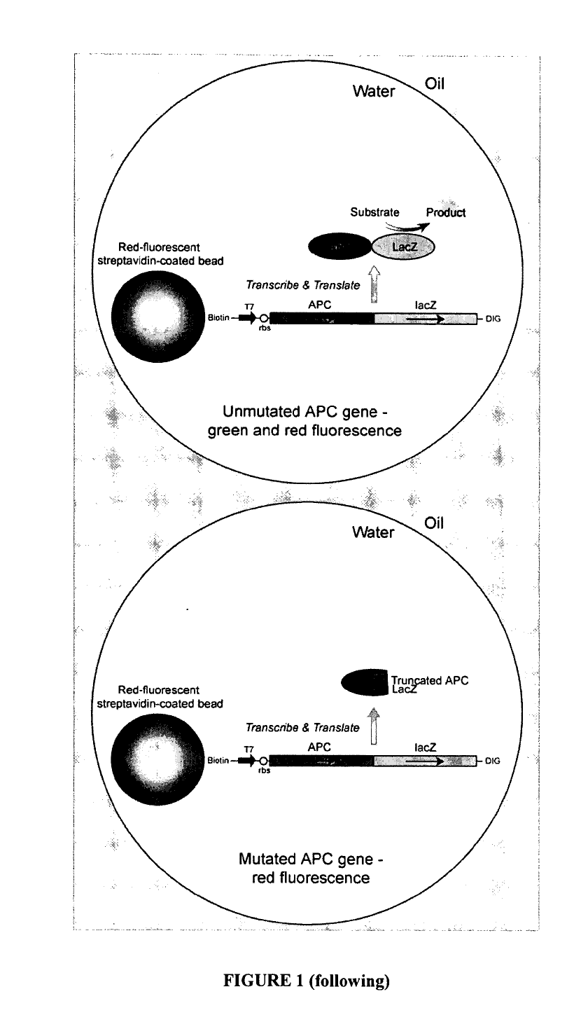

The schematic representation of the method according to the invention used in this example is presented in FIG. 1.

### Example 3

### Detection of Polyps in Stool DNA by Using Genetic Constructs Comprising a Marker Gene as First Reporter System

The MCR of APC genes from stool samples is amplified as described in example 2. One of the PCR primers is used to append a ribosome binding site for translation of the APC gene and a T7 promoter in the reverse orientation. An overlap PCR is then performed to fuse the APC gene in frame with the marker gene lacZ. A second overlap PCR assembles the APC-lacZ fusion gene with the gene encoding a second reporter gene, beta-glucuronidase (GUS) which is in the reverse orientation to APC-lacZ. The DNA fragment carrying the GUS gene carries a ribosome binding site for translation of the beta-glucuronidase and a T7 promoter in the reverse orientation to GUS and in the correct orientation to APC-LacZ. The T7 promoter which is on the GUS DNA fragment drives expression of the APC-lacZ fusion. Hence, there can be no expression of the APC-lacZ fusion gene in the absence of the GUS gene. The T7 promoter which is on the APC DNA fragment drives expression of the GUS fusion. Hence, there can be no expression of the GUS gene in the absence of the APC gene.

Genetic constructs are then mixed with a commercial coupled in vitro transcription and translation system (on ice to prevent the reaction from starting) and dispersed to form a water-in-oil emulsion. The concentration of genetic constructs is set such that very few droplets contain more than one genetic construct. The single genetic construct in each droplet is transcribed and translated.

The activity of the two reporter systems is monitored simultaneously using two highly specific fluorogenic substrates which show negligible cross-reactivity: fluorescein di-beta-D-glucuronide (FDGlcU), which is transformed into fluorescein (green fluorescent) by GUS and resorufin beta-D-galactopyranoside, which transformed into resorufin (orange fluorescent) by LacZ. The droplets are analysed using epifluorescence microscopy and scored using image analysis software.

All droplets containing an APC gene (mutated or unmutated) exhibit GUS activity and become green fluorescent. When the APC gene is unmutated, an APC-LacZ fusion protein is created, there is beta-galactosidase activity and the droplet also becomes orange fluorescent. When the APC gene is mutated, only a truncated APC gene is produced and LacZ is not translated. In this case, there is no beta-galactosidase activity and the droplets are green only. The ratio of green and orange drops, to drops which are green alone gives the percentage of mutated APC genes.

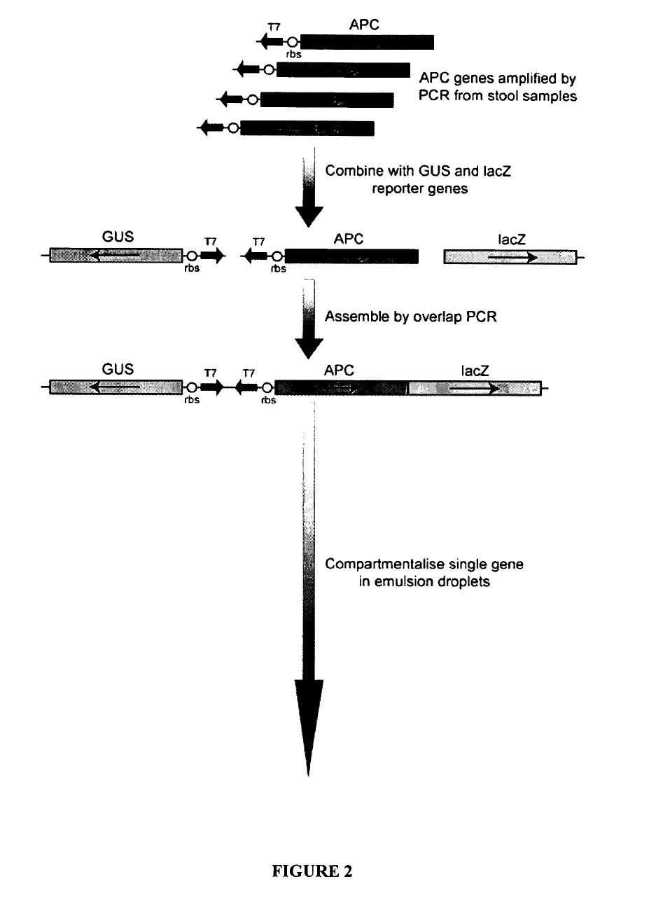

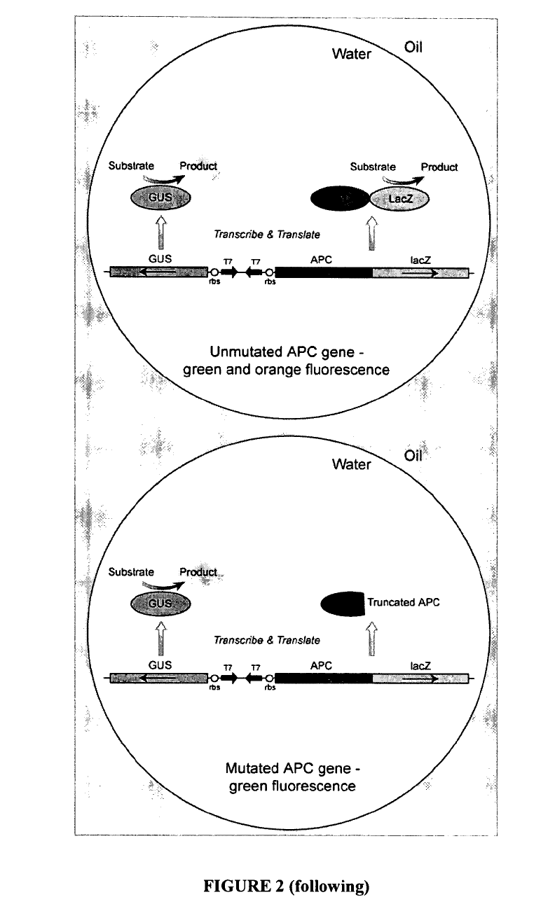

The schematic representation of the method according to the invention used in this example is presented in FIG. 2.

### Example 4

### PCR Amplification within Droplets of an Emulsion

The LacZ gene was inserted in a pIVEX plasmid. pIVEX-LacZ DNA was diluted in 20 ng/μL of carrier tRNA (Ambion) to have a λ˜2. This DNA was added in a PCR mix composed of 1× detergent-free GC buffer (Finnzymes), 200 μM dNTP (MP-Biomedical), 0.5 μM PIVB-4 primer (5′ TTTGGCCGCCGCCCAGT 3′) (SEQ ID No. 1), 0.5 μM LMB10-E primer (5′ GATGGCGCCCAACAGTCC 3′) (SEQ ID No. 2), 3.2 ng/μL modified Picogreen (Raindance Technologies) and 0.02 U Phusion DNA polymerase (Finnzyme). Reaction was emulsified using a microfluidic device in 1.8 μL droplets using fluorocarbon R (RainDance Technologies), containing 2% (w/w) REA surfactant. The emulsion was collected in 0.1 mL PCR tube, covered with mineral oil and thermo-cycled as follow: 30 s at 98° C.; 26 cycles of 10 s at 98° C., 30 s at 55° C., 90 s at 72° C.; and finally 10 min at 72° C.

Mineral oil was then drained out and the green fluorescence of the droplets was analyzed. Droplet fluorescence was monitored using a 488 nm laser and a PMT to collect the light emitted at 525 nm. 100 000 droplets were analyzed.

Results are presented on FIG. 3. The high proportion of positive droplets demonstrated that it is possible to amplify single DNA molecule in droplets through PCR amplification.

### Example 5

### Hyperbranched Rolling Circle Amplification within Droplets of an Emulsion

HRCA commercial kit “Illustra GenomiPhi V2” (G.E Healthcare) and a 6 kb pIVEX-LacZ plasmid bearing the beta-galactosidase coding-gene were used as model system. Amplification mixture according to supplier protocol was further supplemented with 1 μg/μL of purified BSA (New England Biolabs), 2.3 μg/mL of modified Picogreen

(Raindance Technologies) to identify droplets where amplification occurred and 1 mg/μL 70,000 kDa Dextran-Texas Red conjugate (Molecular Probes) as an internal standard. RCA mixtures were discretized in fluorocarbon R oil (RainDance Technologies) containing 2% (w/w) REA surfactant (RainDance Technologies) on a 10 μm nozzle microfluidic device to produce highly monodisperse emulsion of 1.8 μL droplets. Each emulsion was collected in a glass capillary interfaced with the chip by polyethylene tubing and placed 4 hours at 30° C. After incubation, the capillary was connected to an analysis chip, the droplets reinjected and spaced using surfactant-free fluorocarbon R oil. Finally, the fluorescence intensity of droplets pinched on a 10 μm constriction was monitored in an high throughput regime (4-8 kHz) using an optical set-up composed of a 488 nm laser and PMTs measuring light emitted at 525 nm and 585 nm (green and orange fluorescence respectively).

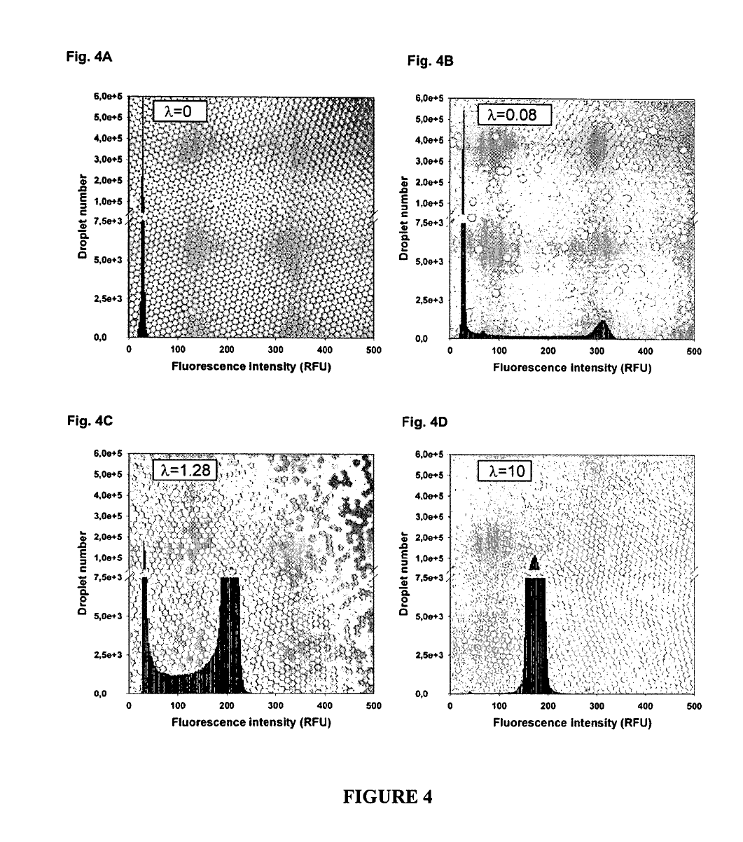

Results are presented on FIG. 4. Single major peaks were obtained with negative (λ=0) (FIG. 4A) and positive (λ=10) controls (FIG. 4D). On the other hand, when using DNA concentrations ranging form 1.5 to 96 pg/μL (λ=0.06 and 1.28 on FIGS. 4B and 4C, respectively), two discrete populations corresponding to inactive (around 25 RFUs) and active droplets (between 200 and 320 RFUs) were distinguishable, demonstrating that it is possible to amplify single DNA molecule in droplets through HRCA.

### Example 6

### Amplification Product-Containing Droplets and IVT Mixture-Containing Droplets Fusion

Amplification product-containing droplets, obtained as described in example 5 with a DNA concentration corresponding to λ=0.25 and Phi29 DNA polymerase, were reinjected in a droplet fusion device and fused with 14 μL volume on-chip generated droplets containing IVT mixture containing 0.7 volume of E. coli extract (EcoProT7, kit, Novagen), 300 μM methionine, 100 μM Fluorescein di-β-D-Galactopyranoside (FDG) (Euromedex) and 1 μM fluorescein.

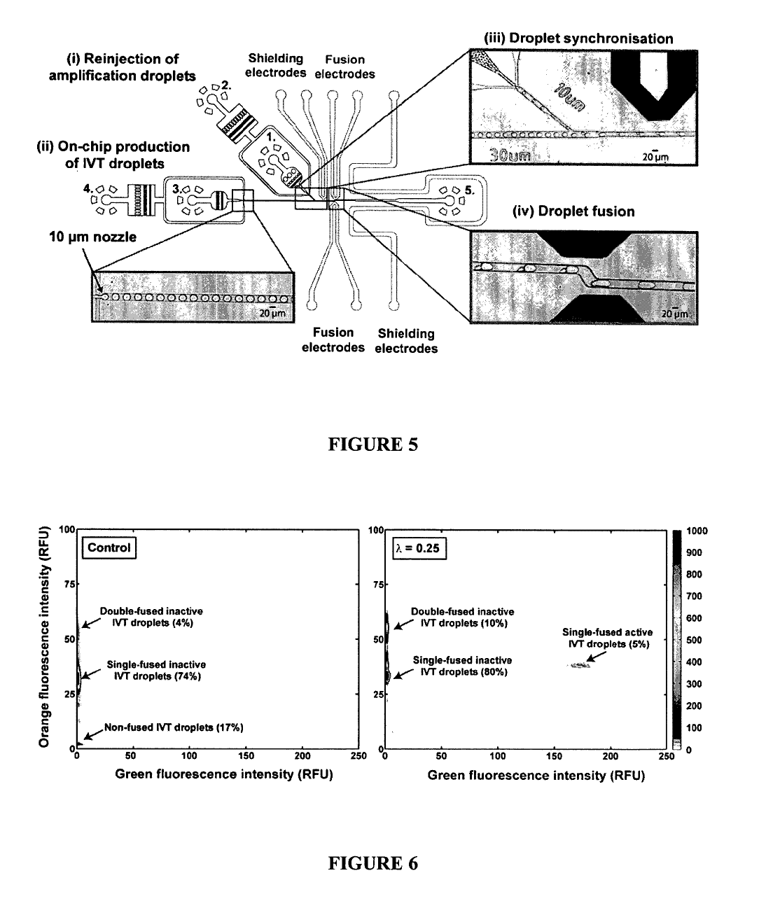

The droplet fusion device consisted of droplet reinjection, on-chip droplet generation, droplet pairing, droplet fusion and droplet collection parts, as presented on FIG. 5. The depth of the PDMS chip was 20 μm. On-chip droplet generation module had a T-shaped junction with 10 μm wide and 15 μm long constriction. Channel down the nozzle was 30 μm wide and 3.5 mm long. The size of the droplet was controlled by adjusting the flow rates of aqueous phase and carrier oil using syringe pumps (PhD Harvard 2000). Droplet reinjection module consisted of T-shaped structure where droplets were spaced by carrier oil. For the electro-coalescence experiments, the inventors had replaced R-oil with FC40 fluorinated liquid (3M®) and increased REA surfactant amount up to 3% (w/w). To increase the spacing between reinjected droplets 10 μm wide channel was used just before merging a pairing channel. A pair of droplets were formed in the pairing channel of 20 μm wide and 800 μm long, just before electro-coalescence region. Electro-coalescence region contained a channel with the turn, where two electrodes were placed perpendicular to the flow direction. An electric field was generated by applying 600 mV ac at 30 kHz across electrodes spaced 120 μm. Finally, after electro-coalescence droplets flowed in the 5.5 mm long exit channel before entering the collection outlet. Shielding electrodes were used in order to prevent non-desirable droplets electro-coalescence during emulsion reinjection and collection.

IVT phase was injected into the droplet generator part of the fusion micro fluidic device at a flow rate of 100 μl/hr and droplets produced by injecting fluorocarbon R containing 2% (w/w) REA surfactant at a flow rate of 100 μl/hr.

Amplification emulsion was reinjected from the capillaries into the fusion device at a flow rate of 20 μl/hr, and the droplets were spaced by detergent-free fluorocarbon R (RainDance Technologies, Lexington, Mass.) at a flow rate of 120 μL/hr. Using these flow rates enabled to pair amplification and IVT droplets with around 80% efficiency. Fusion droplets were collected in a glass capillary and incubated 1 h at 37° C.

Finally, emulsions were reinjected on an analysis device and the fluorescence intensity of each droplet was monitored in a 30 μm wide channel.

The texas red contained in RCA droplets enabled unambiguous identification of single (1 RCA/1 IVT) and double-fused (2 RCA/1 IVT) droplets. On another hand, the green fluorescence resulting from FDG hydrolysis enabled the identification of active droplets where a single plasmid was initially amplified in an HRCA product competent for an expression in protein.

Droplets fluorescence was monitored using a 488 nm and 532 nm lasers (exciting fluorescein and texas red respectively) and photomultiplier tubes (PMT) to collect the light emitted at 525 nm and 610 nm (for fluorescein and texas red respectively). Results of this fluorescence analysis are presented in FIG. 6. The identity of the different populations is given and the percentage of the total population is indicated.

### REFERENCES

Ahn, K., Kerbage, C., Hunt, T., Westervelt, R., Link, D., and Weitz, D. Applied Physics Letters 88 (2006);
Barany (1991) PCR Methods Appl, 1, 5-16;
Becher, P. (1957) Emulsions: theory and practice. Reinhold, New York;
Benita, S. (1996). Microencapsulation: methods and industrial applications. Drugs and pharmaceutical sciences. Edited by Swarbrick, J. New York: Marcel Dekker;
Blanco et al. (1989) J Biol Chem, 264, 8935-8940;
Bonnal et al., Nucleic Acids Res. 2003 Jan. 1; 31(1):427-8;
Cahill et al. (1991) Clin Chem, 37, 1482-1485;
Chetverin and Spirin (1995) Prog Nucleic Acid Res Mol Biol, 51, 225-270;
Christopher and Anna (2007) J. Phys. D: Appl. Phys. 40, R319-R336);
Clausell-Tormos et al. Chem. Biol. 2008 vol. 15 (5) pp. 427-37;
Compton, J. (1991) Nature, 350, 91-92;
Dickinson, E. (1994) In Wedlock, D. J. (ed.), Emulsions and droplet size control. Butterworth-Heine-man, Oxford, pp. 191-257;
Dittrich, P. S, and Schwille, P. Anal Chem 75(21), 5767-5774 (2003);
Eisenstein, M. Nature 441(7097), 1179-1185 (2006);
Embleton, M J, G Gorochov, P T Jones, G Winter. Nucleic Acids Res 1992 vol. (15) pp. 3831-7;
Fahy et al. (1991) PCR Methods Appl, 1, 25-33;
Fidalgo et al. From Microdroplets to Microfluidics: Selective Emulsion Separation in Microfluidic Devices. Angew. Chem. Int. Ed. 2008 vol. 47 (11) pp. 2042-2045;
Fu, A. Y., Spence, C., Scherer, A., Arnold, F. H., and Quake, S. R. Nat Biotechnol 17(11), 1109-1111 (1999);
Fu, A., Chou, H., Spence, C., Arnold, F., and Quake, S. Analytical Chemistry 74, 2451-2457 (2002);
Garvin et al, Eur J Hum Genet, 1998, 6:226-234;
Hein et al. Gene. Hum Mol Genet, 1995, 4:975-981;
Ho, C., Lin, R., Chang, H., and Liu, C. Lab On a Chip 5, 1248-1258 (2005);
Hu, X., Bessette, P. H., Qian, J., Meinhart, C. D., Daugherty, P. S., and Soh, H. T. Proc Natl Acad Sci USA 102(44), 15757-15761 (2005);
Katanaev et al. (1995) Febs Lett, 359, 89-92;
Kelly, B. T., Baret, J.-C., Taly V. and Griffiths A. D. (2007). Miniaturizing chemistry and biology in microdroplets. Chem. Commun., 2007, 1773-1788;
Kim, U., Shu, C.-W., Dane, K. Y., Daugherty, P. S., Wang, J. Y. J., and Soh, H. T. Proceedings of the National Academy of Sciences of the United States of America 104, 20708-20712 (2007);
Kozak, Nature, March 1984, 308, 241-246;
Kruger, J., Singh, K., O'Neill, A., Jackson, C., Morrison, A., and O'Brien, P. Journal of Micromechanics and Microengineering 12, 486-494 (2002);
Landegren et al. (1988) Science, 241, 1077-1080;
Lapizco-Encinas, B. H., Simmons, B. A., Cummings, E. B., and Fintschenko, Y. Electrophoresis 25(10-11), 1695-1704 (2004);
Li et al., Nat. Methods. 2006 February; 3(2):95-7;
Li, P. and Harrison, D. Analytical Chemistry 69, 1564-1568 (1997);
Lissant, K. J., ed. Emulsions and emulsion technology. Surfactant Science New York: Marcel Dekker, 1984;
Lizardi et al. (1998) Nat Genet, 19, 225-232;
Mastrobattista, E., Taly, V., Chanudet, E., Treacy, P., Kelly, B. T., and Griffiths, A. D. Chem Biol 12(12), 1291-1300 (2005);
Notomi et al. (2000) Nucleic Acids Res, 28, E63;
Peral et al. Am J. Hum Genet, 1997, 60:1399-1410;
Perroud, T. D., Kaiser, J. N., Sy, J. C., Lane, T. W., Branda, C. S., Singh, A. K., and Patel, K. D. Anal Chem 80(16), 6365-6372 (2008);
Roest et al. Neuromuscul Disord, 1993, 3:391-394;
Saiki et al. (1988) Science, 239, 481-491;
Sherman, P. (1968) Emulsion science. Academic Press, London;
Shimizu et al. (2001). Nat. Biotechnol. 19, 732-733;
Tawfik and Griffiths, Nature Biotechnol., 1998, 16: 652-656;
Thomas et al., Nature Medicine, 12, 852-855, 2006;
Traverso, et al., New Eng. J. Med, 346, 311-320, 2002;
Walker et al. (1992) Nucleic Acids Res, 20, 1691-1696;
Wang, M. M., Tu, E., Raymond, D. E., Yang, J. M., Zhang, H., Hagen, N., Dees, B., Mercer, E. M., Forster, A. H., Kariv, I., Marchand, P. J., and Butler, W. F. Nat Biotechnol 23(1), 83-87 (2005);
Wolff, A., Perch-Nielsen, I., Larsen, U., Friis, P., Goranovic, G., Poulsen, C., Kutter, J., and Telleman, P. Lab On a Chip 3, 22-27 (2003).

1. A method for detecting or quantifying a truncating mutation of a target gene in a subject, said method relying on the in vitro compartmentalization of single genetic constructs in aqueous droplets of a water-in-oil emulsion and comprising:
a) providing a DNA sample from the subject;
b) assembling genetic constructs, each construct comprising a test sequence of said target gene, obtained from said DNA sample, operably linked with a promoter and a ribosome binding site, a first reporter system used to control the presence of said test sequence in the construct and/or the presence of a construct comprising said test sequence in a droplet, and a second reporter system used to detect the presence of a truncating mutation of said target gene, said first and second reporter systems generating distinct signals;
c) compartmentalizing each genetic construct in a droplet by forming a water-in-oil emulsion;
d) transcription and translation of each genetic construct in each droplet
e) monitoring emitted signals from said first and second reporter systems in each droplet to detect or quantify truncating mutations of said target gene.

2. The method according to claim 1, further comprising an amplification step of said test sequence from said DNA sample before assembling genetic constructs.

3. The method according to claim 2, wherein said amplification step is performed by using the polymerase chain reaction.

4. The method according to claim 2, wherein said amplification step is performed by using Hyperbranched Rolling Circle Amplification.

5. The method according to any one of claims 1 to 4, wherein said genetic constructs are compartmentalized together with an in vitro transcription and translation system.

6. The method according to any one of claims 1 to 5, wherein said genetic constructs are compartmentalized together with one or several substrates necessary to generate reporter system signals.

7. The method according to any one of claims 1 to 6, wherein the second reporter system is a marker gene operably linked to said test sequence in order to be expressed in a single mRNA, said marker gene being downstream to said test sequence.

8. The method according to claim 7, wherein said marker gene is fused in frame with said test sequence.

9. The method according to any one of claims 1 to 8, wherein the first reporter system is a marker gene expressed on a polycistronic mRNA further comprising the test sequence fused in frame with the marker gene of the second reporter system and an internal ribosome entry site or an internal ribosome binding site, said test sequence being downstream to said first reporter system and upstream to said second reporter system, and said internal ribosome entry site or internal ribosome binding site being operably linked to said test sequence.

10. The method according to any one of claims 1 to 8, wherein the first reporter system is a marker gene expressed from a promoter which is operably linked with said marker gene only if said test sequence is present in the construct.

11. The method according to any one of claims 1 to 10, wherein said first and second reporter systems are different and are selected from the group consisting of beta-galactosidase, beta-glucuronidase, beta-glucosidase, luciferase, horseradish peroxidase, alkaline phosphatase, green fluorescent protein, DsRed, Keima and derivatives thereof.

12. The method according to any one of claims 1 to 11, wherein said genetic constructs are amplified before compartmentalization.

13. The method according to any one of claims 1 to 12, wherein said genetic constructs are amplified after compartmentalization of step c) and wherein said method further comprises, after said amplification, an additional step of fusing droplets containing said amplified genetic constructs with droplets containing an in vitro transcription and translation system.

14. The method according to any one of claims 1 to 8, wherein the first reporter system is an affinity system comprising two members, a first member appended on the 5′ end of the coding strand of said test sequence and a second member which is able to generate a signal and to bind said first member.

15. The method according to claim 14, further comprising an additional step before c) to remove said second members which are not bound to said first members.

16. The method according to claim 15, wherein the additional step is an affinity purification involving a digoxinenin tag on the genetic construct and non-fluorescent magnetic beads coated with an anti-digoxinenin antibody.

17. The method according to any one of claims 14 to 16, wherein said first member is a biotin tag and said second member is a fluorescent steptavidin coated bead.

18. A method for detecting or quantifying a truncating mutation of a target gene in a subject, said method relying on the in vitro compartmentalization of single genetic constructs in aqueous droplets of a water-in-oil emulsion and comprising:
a) providing a DNA sample from the subject;
b) compartmentalizing each DNA molecule, from said DNA sample, comprising a test sequence of said target gene in first droplets by forming a water-in-oil emulsion;
c) assembling genetic constructs, each construct comprising said test sequence operably linked with a promoter and a ribosome binding site, a marker gene of a first reporter system used to control the presence of said test sequence in the construct and/or the presence of a construct comprising said test sequence in a droplet, and a marker gene of a second reporter system used to detect the presence of a truncating mutation of said target gene, said first and second reporter systems generating distinct signals;
d) fusing first droplets containing genetic constructs of step c) with second droplets containing an in vitro transcription and translation system;
e) transcription and translation of each genetic construct in each fusion droplet
f) monitoring emitted signals from said first and second reporter systems in each fusion droplet to detect or quantify truncating mutations of said target gene.

19. The method according to claim 18, further comprising an amplification step of said test sequence from said DNA sample before step b).

20. The method according to claim 18, further comprising an amplification step of said test sequence from said DNA molecules contained into droplets after step b) and before step c).

21. The method according to claim 19 or 20, wherein said amplification step is performed by using the polymerase chain reaction.

22. The method according to claim 19 or 20, wherein said amplification step is performed by using Hyperbranched Rolling Circle Amplification.

23. The method according to any one of claims 18 to 22, wherein said first and/or second droplets further contain one or several substrates necessary to generate reporter system signals.

24. The method according to any one of claims 18 to 23, wherein said marker gene of the second reporter system is operably linked to said test sequence in order to be expressed in a single mRNA, said marker gene being downstream to said test sequence.

25. The method according to claim 24, wherein said marker gene is fused in frame with said test sequence.

26. The method according to any one of claims 18 to 25, wherein the marker gene of the first reporter system is expressed on a polycistronic mRNA further comprising the test sequence fused in frame with the marker gene of the second reporter system and an internal ribosome entry site or an internal ribosome binding site, said test sequence being downstream to said first reporter system and upstream to said second reporter system, and said internal ribosome entry site or internal ribosome binding site being operably linked to said test sequence.

27. The method according to any one of claims 18 to 25, wherein the marker gene of the first reporter system is expressed from a promoter which is operably linked with said marker gene only if said test sequence is present in the construct.

28. The method according to any one of claims 18 to 27, wherein marker genes of said first and second reporter systems are different and are selected from the group consisting of beta-galactosidase, beta-glucuronidase, beta-glucosidase, luciferase, horseradish peroxidase, alkaline phosphatase, green fluorescent protein, DsRed, Keima and derivatives thereof.

29. The method according to any one of claims 18 to 28, wherein said marker gene of the first reporter system is the beta-glucuronidase encoding gene and said marker gene of the second reporter system is the beta-galactosidase encoding gene.

30. The method according to any one of claims 18 to 29, wherein said genetic constructs are amplified before step d).

31. The method according to any one of claims 1 to 30, further comprising an additional step after step e) of claim 1 and step f) of claim 17 of sorting the droplets to allow further characterisation or manipulation of said test sequence.

32. The method according to any one of claims 1 to 31, wherein the detection or quantification of truncating mutations of a target gene in a subject is used to diagnose or prognosticate a disease.

33. The method according to claim 32, wherein the target gene is a tumor suppressor gene.

34. The method according to claim 32, wherein said disease is selected from the group consisting in a colorectal cancer, a breast cancer, an ovarian cancer, a polycystic kidney disease, a neurofibromatosis and a Duchenne muscular dystrophy.

35. The method according to any one of claims 32 to 34, wherein said disease is a colorectal cancer.

36. The method according to claim 35, wherein said DNA sample is obtained from a stool sample.

37. The method according to claim 35 or 36, wherein said target gene is the APC gene.

38. The method according to claim 37, wherein said test sequence is the MCR of APC gene.

39. A genetic construct comprising a test sequence operably linked with a promoter and a ribosome binding site, a first marker gene operably linked with another promoter, and a second marker gene which is operably linked to the test sequence in order to be expressed in a single mRNA, said marker gene being downstream to said test sequence.

40. The genetic construct according to claim 39, wherein said first and second marker genes are different and are selected from the group consisting of genes encoding beta-galactosidase, beta-glucuronidase, beta-glucosidase, luciferase, horseradish peroxidase, alkaline phosphatase, green fluorescent protein, DsRed, Keima and derivatives thereof.

41. The genetic construct according to claim 39 or 40, wherein said first marker gene is the beta-glucuronidase encoding gene and said second marker is the beta-galactosidase encoding gene.

42. A genetic construct comprising a test sequence operably linked with a promoter and a ribosome binding site, a first reporter system which is an affinity system comprising two members, a first member appended on the 5′ end of the coding strand of the test sequence and a second member which is bound to the first member and is able to generate a signal, and a marker gene which is operably linked to the test sequence in order to be expressed in a single mRNA, said marker gene being downstream to said test sequence

43. The genetic construct according to claim 42, wherein said first member is a biotin tag and said second member is a fluorescent steptavidin coated bead.

44. The genetic construct according to claim 42 or 43, wherein the marker gene is selected from the group consisting of genes encoding beta-galactosidase, beta-glucuronidase, beta-glucosidase, luciferase, horseradish peroxidase, alkaline phosphatase, green fluorescent protein, DsRed, Keima and derivatives thereof.

45. The genetic construct according to claim 44, wherein the marker gene is the beta-galactosidase encoding gene

46. The genetic construct according to any one of claims 39 to 45, wherein the test sequence comprises all or part of the APC gene.

47. A droplet from a water-in-oil emulsion containing a genetic construct comprising at least two reporter systems generating distinct signals.

48. A Kit for the detection or quantification of a truncating mutation in a target gene in a subject by using the method according to any one of claims 1 to 38, comprising at least reagents needed to assemble genetic constructs of claims 39 to 46, an in vitro transcription/translation system, reagents needed to form a water-in-oil emulsion and, optionally means needed to compartmentalize each genetic construct or DNA molecule into droplets.

49. The kit according to claim 48, further comprising primers suitable for amplifying the test sequence of the target gene and/or primers suitable for assembling the genetic construct.

50. The kit according to claim 48 or 49, wherein reagents needed to assemble genetic constructs of claims 40 to 46 comprise a plasmid containing the marker gene of the first reporter system and/or the marker gene of the second reporter system

51. The kit according to any one of claims 48 to 50, further comprising one or more substrates needed to generate reporter system signals.

52. The Kit according to any one of claims 48 to 51, further comprising one or more surfactants.
